(19) United States
(12) Patent Application Publication
(10) Pub. No.: US 2022/0415457 A1
(43) Pub. Date: Dec. 29, 2022

(54) METHOD AND SYSTEM FOR MEDICAL SUPPORT

(71) Applicant: TOPCON CORPORATION, Tokyo (JP)

(72) Inventor: Noriyasu TAKEDA, Tokyo (JP)

(73) Assignee: TOPCON CORPORATION, Tokyo (JP)

(21) Appl. No.: 17/823,082

(22) Filed: Aug. 30, 2022

Related U.S. Application Data
(63) Continuation of application No. 15/634,170, filed on Jun. 27, 2017.

(30) Foreign Application Priority Data
Jul. 1, 2016 (JP) 2016-131617

(51) Int. Cl. G16H 10/60; G16H 50/20; G16H 40/20

(52) U.S. Cl. CPC G16H 10/60; G16H 50/20; G16H 40/20

(57) ABSTRACT

A computer-based method for medical support. A computer receives history data obtained from history taking of a patient, selects one or more first examination types for a first examination from a first examination type group stored in a storage device in advance based on the history data, and controls an output device to output information indicating the one or more first examination types. Further, the computer receives first examination data obtained from the first examination of the patient, wherein the first examination includes at least one of the one or more first examination types, selects one or more second examination types for a second examination from a second examination type group stored in the storage device in advance based on the history data and the first examination data, and controls the output device to output information indicating the one or more second examination types.

### CROSS-REFERENCE TO RELATED APPLICATION

This application is a Continuation of U.S. patent application Ser. No. 15/634,170, filed Jun. 27, 2017, which is based upon, and claims the benefit of priority from, Japanese Patent Application No. 2016-131617, filed Jul. 1, 2016; the entire contents of all of which are incorporated herein by reference.

### BACKGROUND

In medical care, diagnosis is carried out based on information obtained from history taking (or, inquiry, interview, or the like) and examinations, and a treatment plan is determined. Typically, history taking is firstly performed to obtain information, and types of examinations are determined based on the information. Then, diagnosis is carried out with referring to the information obtained by the history taking and the examinations.

Examples of information obtained from history taking include chief complaint, present history, past history, family history, life history, occupational history, travel history, and the like. Types of examinations includes measurements and imaging. There are various kinds of examinations according to diagnosis and treatment departments. For example, in the ophthalmology department, subjective refraction measurement, objective refraction measurement, tonometry, slit lamp microscope examination, eye fundus photography, optical coherence tomography (OCT), visual field examination, and the like can be listed as typical examinations. In addition, various kinds of methods are used to analyze information obtained from examinations. Typical examples of such analysis methods in ophthalmology include tissue morphology analysis based on data acquired using OCT (e.g., retinal thickness analysis and optic nerve head morphology analysis).

Application of computer technologies such as artificial intelligence to medical support has made rapid strides. Typical examples of the application include expert systems, assistance for interaction with patients etc., assistance for formulating treatment plans, image processing, and the like.

### PATENT DOCUMENTS

Japanese Unexamined Patent Application Publication No. 2009-211714

Japanese Unexamined Patent Application Publication No. 2015-167863

Japanese Unexamined Patent Application Publication No. 2010-020784

Japanese Unexamined Patent Application Publication No. 2015-154918

Japanese Unexamined Patent Application Publication (Translation of PCT Application) No. 2015-501667

In order to improve accuracy of diagnosis, it is desirable to take a lot of information into account. In order to obtain a lot of information, it is required to prolong time for communication with a patient, or to increase types of examinations to be performed, which increases time and burdens required for diagnosis or examination. Therefore, a burden on a patient can be increased, a burden on medical workers can be increased, and efficiency of diagnosis and treatment can be deteriorated.

In particular, in the case where a person other than medical professionals (e.g., doctors) carries out an examination like home health care or medical checkup vehicles, it is very difficult to determine types of examinations to be further performed based on information obtained from history taking and examinations, and to determine a possible disease. Hence, the above problems are more serious.

### BRIEF SUMMARY

The first aspect of a medical support method according to an exemplary embodiment is a computer-based method for medical support. The computer performs the steps of: receiving history data obtained from history taking of a patient; selecting one or more first examination types for a first examination from a first examination type group stored in a storage device in advance based on the history data; controlling an output device to output information indicating the one or more first examination types; receiving first examination data obtained from the first examination of the patient, wherein the first examination includes at least one of the one or more first examination types; selecting one or more second examination types for a second examination from a second examination type group stored in the storage device in advance based on the history data and the first examination data; and controlling the output device to output information indicating the one or more second examination types.

The second aspect of a medical support method according to an exemplary embodiment is a computer-based method for medical support, wherein a computer performs the steps of: receiving history data obtained from history taking of a patient; receiving examination data obtained from an examination of the patient; selecting one or more examination types for another examination from an examination type group stored in a storage device in advance based on the history data and the examination data; and controlling an output device to output information indicating the one or more examination types.

The first aspect of a medical support system according to an exemplary embodiment includes: a storage unit configured to store a first examination type group and a second examination type group in advance; a history data reception unit configured to receive history data obtained from history taking of a patient; a first selection unit configured to select one or more first examination types for a first examination from the first examination type group based on the history data; an output unit; an output controller configured to control the output unit to output information indicating the one or more first examination types; an examination data reception unit configured to receive first examination data obtained from the first examination of the patient, wherein the first examination includes at least one of the one or more first examination types; and a second selection unit configured to select one or more second examination types for a second examination from the second examination type group based on the history data and the first examination data, wherein the output controller controls the output unit to output information indicating the one or more second examination types.

The second aspect of a medical support system according to an exemplary embodiment includes: a storage unit configured to store a first examination type group and a second examination type group in advance; a history data reception unit configured to receive history data obtained from history taking of a patient; a first selection unit configured to select one or more first examination types for a first examination from the first examination type group based on the history data; an output controller configured to control an output device to output information indicating the one or more first examination types; an examination data reception unit configured to receive first examination data obtained from the first examination of the patient, wherein the first examination includes at least one of the one or more first examination types; and a second selection unit configured to select one or more second examination types for a second examination from the second examination type group based on the history data and the first examination data, wherein the output controller controls the output device to output information indicating the one or more second examination types.

### BRIEF DESCRIPTION OF THE DRAWINGS

FIG. 1 is a flow chart illustrating an exemplary medical support method.

FIG. 2 is a schematic diagram illustrating the configuration of an exemplary medical support system.

FIG. 3 is a schematic diagram illustrating examination type information used for the selection of an examination type executed by the exemplary medical support system.

FIG. 4 is a flow chart illustrating a usage mode that can be performed using the exemplary medical support system.

FIG. 5 is a schematic diagram illustrating information displayed in the exemplary usage mode.

FIG. 6 is a schematic diagram illustrating information displayed in the exemplary usage mode.

FIG. 7 is a schematic diagram illustrating information displayed in the exemplary usage mode.

### DETAILED DESCRIPTION

Exemplary embodiments of the present invention will be described in detail with referring to the drawings. Exemplary medical support methods can be realized with exemplary medical support systems. An exemplary medical support system may include two or more apparatuses capable of communicating with one another. For example, such an exemplary medical support system includes one or more computers and one or more storage devices. Alternatively, an exemplary medical support system may be a single apparatus such as a computer including a storage device.

Hardware and software for implementing exemplary medical support methods are not limited to those of the exemplary medical support systems described below. Arbitrary combination of any hardware and any software for the implementation can be included in an exemplary medical support system, A medical support system of a typical example may include hardware and software that function as an artificial intelligence engine.

The medical support methods are utilized for performing medical examinations. More specifically, the medical support methods are utilized for determining types of examinations to be performed on patients. Examination types are determined, for example, based on at least data obtained by performing history taking on a patient (referred to as history data). In addition, data obtained by an examination carried out in the past (referred to as examination data) may be used to determine the examination types. Hereinafter, examples of cases where a medical support method is applied to the ophthalmology field are mainly described. However, ophthalmology is not the only medical field to which the medical support method is applied. The medical support method can be applied to any other medical field.

### <Examples of Medical Support Methods>

In the present example, the medical support method is applied to the ophthalmology field. The medical support method can be operated in or outside a medical institution. Examples of operation locations outside the medical institution include a medical checkup vehicle, a patient's home, a nursing home, a welfare facility, a drug store, an optician's store, and the like. Systems, apparatuses, devices, and the like for implementing the medical support method will be described later.

Processing according to the medical support method is executed by a computer(s). The computer may be configured to function as an artificial intelligence engine. The computer includes one or more processors. The processor is a circuit such as a central processing unit (CPU), a graphics processing unit (GPU), an application specific integrated circuit (ASIC), a programmable logic device (e.g., simple programmable logic device (SPLD), complex programmable logic device (CPLD)), a field programmable gate array (FPGA), or the like. The processor is configured, for example, to read out a computer program from a storage device (or a storage circuit), and to execute the computer program, thereby performing a desired function.

The processor may be configured to control the storage device and/or an output device. The storage device may be included in the computer or may be arranged outside the computer. Similarly, the output device may be arranged in or outside the computer.

The storage device stores information for executing the selection of examination types. For example, the storage device stores a first examination type group. The first examination type group includes options of examination types for the first examination. The first examination is carried out based on the history data. The storage device stores a second examination type group. The second examination type group includes options of examination types for the second examination. The second examination is carried out based on the history data and the first examination data (i.e., the result obtained by the first examination). The storage device stores a third examination type group. The third examination type group includes options of examination types for the third examination. The third examination is carried out based on the history data, the first examination data, and the second examination data (i.e., the result obtained by the second examination).

The output device is a device for outputting information. A typical example of the output device is a display device, an audio output device, a printer, a communication device having data transmission function, a data writer that records information in a recording medium, or the like. Any one or more of these exemplary devices are included in the output device

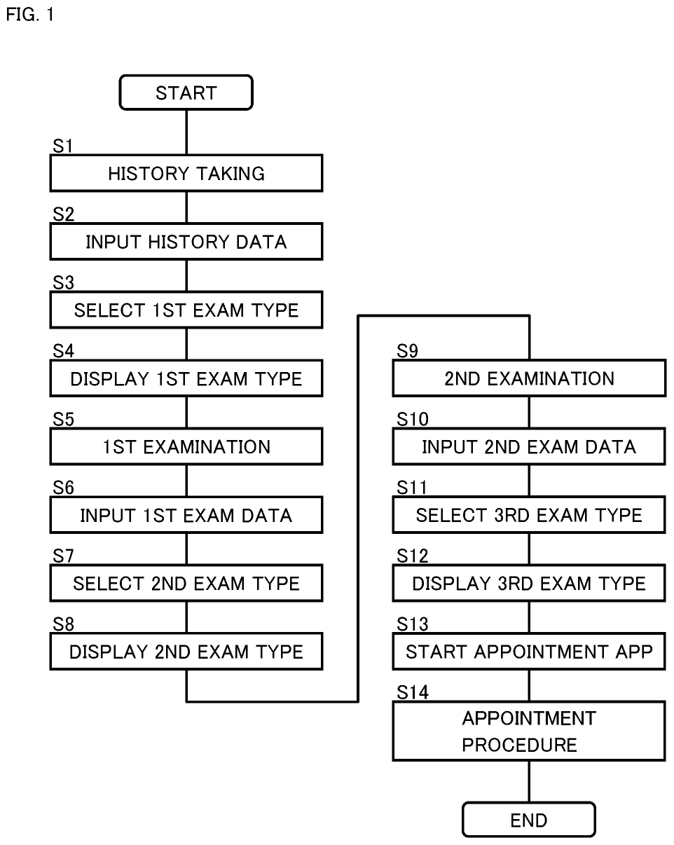

FIG. 1 shows an example of the medical support method. Patient registration etc. can be performed prior to the step 105 (also referred to herein as “S1”) of FIG. 1. Here, the patient means a person to be examined. Hence, the patient is not limited to a person who already has consulted a medical institution, and may be a person who has not consulted a medical institution yet (e.g., a person who is going to undergo an examination for the screening for a specific disease). The timing of performing the patient registration is not necessarily before the step S1, and may be any timing during the execution of the step S1, or may be any timing after the step S1.

### (S1: History Taking)

To begin with, at S1, history taking of the patient is carried out. In the history taking, inquiry concerning various items such as chief complaint, present history, past history, family history, life history, occupational history, and travel history is made to the patient, and the patient gives answers to the inquiry. The history taking can be carried out in any way. Examples of the history taking method will be described in the following. Note that the history taking methods are not so limited.

In one example, a computer controls the display device to display a screen for history taking (referred to as a history taking screen). The history taking screen is provided with inquiry and answer entering spaces. The patient operates an input device (e.g., keyboard, mouse, touch panel) to enter answers in the answer entering spaces. Examples of the answer entering mode include text entry (or character entry), selection from a plurality of options (e.g., using check boxes, drop down menus).

In another example, a person other than the patient (e.g., the examiner) gives inquiry and/or enters answers. The mode of outputting the inquiry is not limited to displaying; the inquiry can be output with voice or the like. Similarly, the mode of entering answers is not limited to displaying; the answer can be entered with voice or the like. In such a case, voice recognition technology can be employed to transform the answers as audio data to character data.

In yet another example, answers to inquiry is input on a paper sheet (referred to as a history taking sheet). Typically, the history taking sheet is provided with inquiry and answer entering spaces. The patient uses a pen etc. to write answers in the answer entering spaces. Alternatively, a person other than the patient (e.g., the examiner) can give inquiry and/or write answers.

### (S2: Input History Data)

The computer is fed with the history data, at step 110 (also referred to herein as “S2”), obtained in the step S1. The mode of inputting the history data into the computer is arbitrary. For example, when the answers are entered in the computer in the step S1, the input digital data or data obtained by processing the input digital data is used. When the computer into which the answers has been entered in the step S1 executes the processes of the step S3 and the following steps, entering the answers in the step S1 and inputting the answers in the step S2 are the same. When the computer (referred to as a computer for history taking) into which the answers has been entered in the step S1 and the computer (referred to as a computer for processing) that executes the processes of the step S3 and the following steps are different from each other, the answers entered into the computer for history taking (i.e., history data) is directly or indirectly transmitted to the computer for processing.

### (S3: Select First Examination Type)

Based on the history data received in the step S2, the computer selects one or more first examination types, at step 115 (also referred to herein as “S3”), for the first examination from the first examination type group stored in the storage device in advance.

The process of the step S3 may be executed using the artificial intelligence engine. For example, when the history data includes information represented by a natural language, the artificial intelligence engine applies predetermined natural language processing to the history data received in the step S2. The computer (e.g., the artificial intelligence engine) can select a first examination type based on the result obtained by the natural language processing.

In another example, it is possible to prepare table information in which a first examination type(s) is associated with each of combinations of answers to history taking items. With referring to the table data, the computer can select a first examination type according to the history data received in the step S2.

### (S4: Display First Examination Type, Etc.)

The computer controls the output device to output information indicating the first examination type, at step 120 (also referred to herein as “S4”), selected in the step S3. When the output device includes the display device, the computer can control the display device to display information indicating the first examination type selected in the step S3 (e.g., a character string representing the examination type, a list of the examination types, etc.). When the output device includes the audio output device, the computer can control the audio output device to output audio information indicating the first examination type selected in the step S3 (e.g., synthesized voice). When the output device includes the printer, the computer can control the printer to output information indicating the first examination type selected in the step S3 (e.g., a character string representing the examination type, a list of the examination types, etc.) on a printing paper. When the output device includes the communication device, the computer can control the communication device to transmit information indicating the first examination type selected in the step S3 to a predetermined apparatus (e.g., server, archiving system, mobile terminal). When the output device includes the data writer, the computer can control the data writer to record information indicating the first examination type selected in the step S3 on a recording medium.

### (S5: First Examination)

The first examination is carried out at step 125 (also referred to herein as “S5”). The first examination includes (at least one of) the first examination types output in the step S4. The examination is carried out according to instructions from the examiner or the examination apparatus. When two or more examinations are carried out, the order of the examinations is determined in any manner. For example, the computer may be configured to determine the examination order according to operation states (e.g., time required for examination, the number of waiting patients, or the like) of various kinds of examination apparatuses.

### (S6: Input First Examination Data)

The computer is fed with the data, at step 130 (also referred to herein as “S6”), obtained from the first examination carried out in the step S5 (referred to as first examination data). The mode of inputting the first examination data is arbitrary. In one example, when the first examination is carried out using an examination apparatus that can communicate with the computer, the first examination data can be transmitted form the examination apparatus to the computer. In another example, the first examination data obtained by an examination apparatus can be recorded in a recording medium, and input from the recording medium to the computer using a data reader provided in the computer.

### (S7: Select Second Examination Type)

Based on the history data received in the step S2 and the first examination data received in the step S6, the computer selects one or more second examination types, at step 135 (also referred to herein as “S7”), for the second examination from the second examination type group stored in the storage device in advance. This selection process can be executed, for example, using the artificial intelligence engine or the table information as in the step S3.

### (S8: Display Second Examination Type, Etc.)

The computer controls the output device to output, at step 140 (also referred to herein as “S8”), information indicating the second examination type selected in the step S7. This output process can be executed in the same manner as in the step S4.

### (S9: Second Examination)

The second examination is carried out at step 145 (also referred to herein as “S9”). The second examination includes (at least one of) the first examination types output in the step S8. The second examination can be carried out in the same manner as in the step S5.

### (S10: Input Second Examination Data)

The computer is fed with the data, at step 150 (also referred to herein as “S10”), obtained from the second examination carried out in the step S9 (referred to as second examination data). This input process can be executed in the same manner as in the step S6.

### (S11: Select Third Examination Type)

Based on the history data received in the step S2, the first examination data received in the step S6, and the second examination data received in the step S10, the computer selects one or more third examination types, at step 155 (also referred to herein as “S11”), for the third examination from the third examination type group stored in the storage device in advance. This selection process can be executed, for example, using the artificial intelligence engine or the table information as in the step S3.

### (S12: Display Third Examination Type, Etc.)

The computer controls the output device to output, at step 160 (also referred to herein as “S12”), information indicating the third examination type selected in the step S11. This output process can be executed in the same manner as in the step S4.

### (S13: Start Appointment Application)

For example, in the case where the third examination cannot be carried out in the current examination location, or in the case where the patient should consult a specialist, the computer starts up application software for appointment, at step 165 (also referred to herein as “S13”), to consult a specialized medical institution or an advanced medical institution.

The application software is, for example, stored in the storage device or transmitted from another computer as needed. Alternatively, it may be configured that the computer is used as user interface for the appointment processing while another computer (e.g., server) executes the appointment processing.

### (S14: Appointment Procedure)

When the appointment application is activated, the computer controls the display device in the output device to display an appointment screen, for example. In the appointment screen, at step 170 (also referred to herein as “S14”), various information such as patient information (e.g., name, sex, date of birth, social insurance number, patient ID, email address, etc.) and the list of appointable medical institutions (e.g., specialized medical institutions etc.) is displayed. In addition, the appointment screen is provided with objects for selecting whether the patient hopes to make an appointment or not (e.g., drop down menus, check boxes, etc.), objects for designating preferred appointment dates, and the like.

Appointable dates of the respective medical institutions can be displayed on the appointment screen. In such a case, the computer can obtain information (e.g., states of appointments) from in-hospital servers of the respective medical institutions or from a server communicable with the in-hospital servers, for example.

The user (e.g., patient, examiner) enters predetermined necessary information into the appointment screen using an operation device. The entered information is directly or indirectly transmitted to the in-hospital server of the medical institution designated. When the appointment processing is completed, information indicating the completion of appointment is directly or indirectly transmitted from the in-hospital server to the computer. It is also possible to transmit information indicating the completion of appointment to the patient's email address from the computer, the in-hospital server or another computer.

### <Medical Support System>

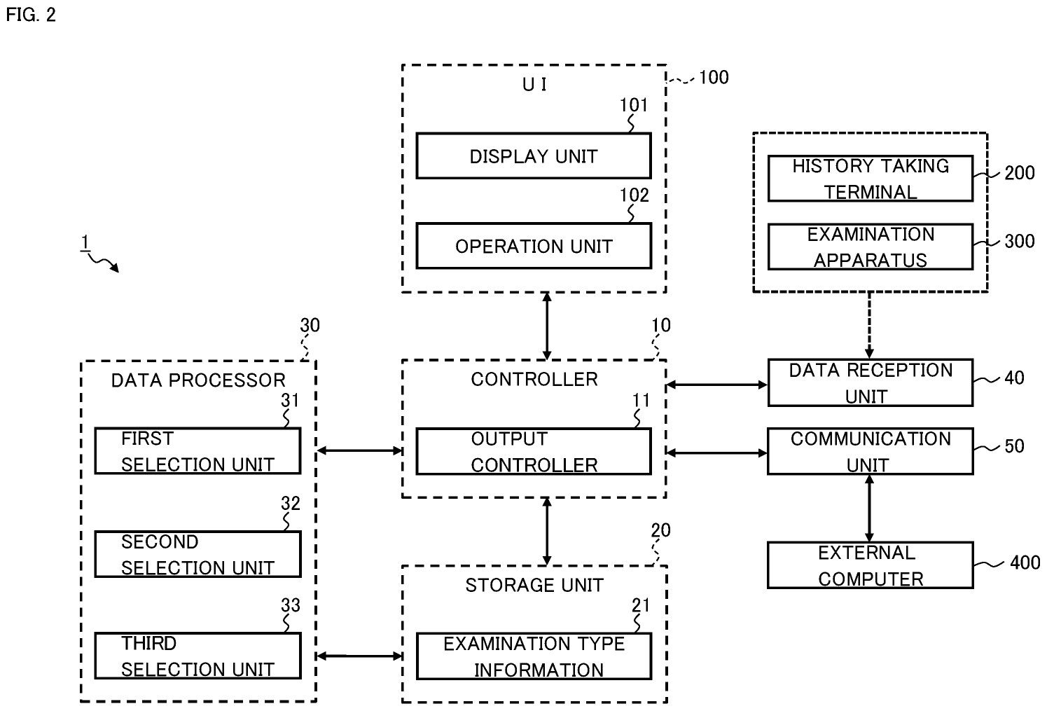

A system for implementing the medical support method described above will be described. FIG. 2 shows an example of the configuration of such a system.

The medical support system 1 includes the controller 10, the storage unit 20, the data processor 30, the data reception unit 40, the communication unit 50. The user interface (UI) 100 may or may not be included in the medical support system 1. Components included in the medical support system 1 are configured to be a single apparatus or two or more apparatuses. For example, the medical support system 1 includes a single computer that is provided with all the components.

In one example in which the medical support system 1 includes two or more apparatuses, a computer including the controller 10, a computer including the storage unit 20, and a computer including the data processor 30 are arranged separately. In another example, the medical support system 1 includes a computer including any two of the controller 10, the storage unit 20, and the data processor 30, and a computer including the remaining one. The communication mode between the different computers may include wired communication and/or wireless communication, may include a private line and/or a public line, and may include at least one of a local area network (LAN), a wide area network (WAN), near field communication, and the internet.

### <Controller 10>

The controller 10 executes various kinds of control. For example, the controller 10 executes control of each component of the medical support system 1 and linkage control (or interlock control) of two or more components. The controller 10 can execute control of an external apparatus arranged outside the medical support system 1. For example, when the user interface 100 is not included in the medical support system 1, the controller 10 can executes control of the user interface 100. The controller 10 includes a processor.

### <Output Controller 11>

The output controller 11 executes control of the output device described above. In the present example, the output controller 11 executes the step S4 (display first examination type, etc.), the step S8 (display second examination type, etc.), and the step S12 (display third examination type, etc.) in FIG. 1.

For example, the output device includes at least one of the display device, the audio output device, the printer, the communication device, and the data writer. In such a case, the output controller 11 is configured to execute any of the following control: control of the display device for displaying information; control of the audio output device for outputting audio information; control of the printer for printing information on a printing paper; control of the communication device for sending information to an external apparatus; and control of the data writer for recording information in a recording medium.

In the example shown in FIG. 2, the display unit 101 is provided as the display device. The communication unit 50 may be used as the communication device. Although illustration is omitted, one or more of the audio output device, the printer, and the data writer may be included in the medical support system 1. The medical support system 1 may include other kinds of output devices.

### <Storage Unit 20>

The storage unit 20 stores various kinds of data. Examples of data stored in the storage unit 20 include patient information such as a patient's name and patient ID. The storage unit 20 includes, for example, at least one of a semiconductor storage, a magnetic storage, an optical storage, and a magneto-optical storage.

### <Examination Type Information 21>

The storage unit 20 stores the examination type information 21 in advance. The examination type information 21 is used for selecting examination types, and includes the first examination type group, the second examination type group, and the third examination type group described above. Here, the first examination type group includes options of examination types for the first examination that is carried out based on the history data. The second examination type group includes options of examination types for the second examination that is carried out based on the history data and the first examination data (i.e., the result obtained by the first examination). The third examination type group includes options of examination types for the third examination that is carried out based on the history data, the first examination data, and the second examination data (i.e., the result obtained by the second examination).

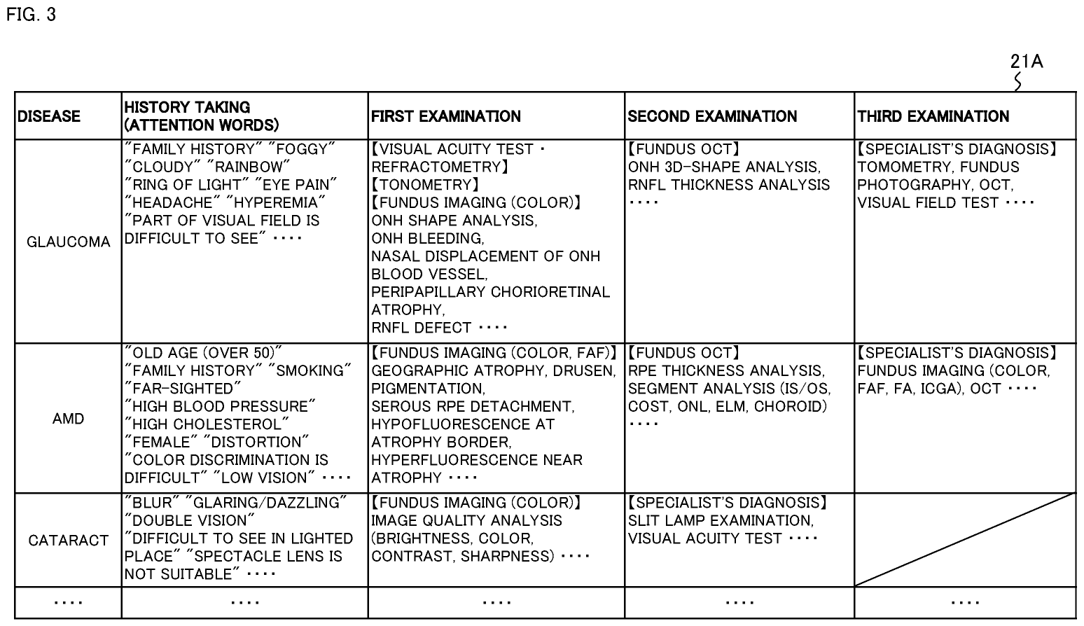

FIG. 3 shows an example of the examination type information 21. The examination type information 21A is table information in which various kinds of information is associated with each of a plurality of diseases that are targets of medical screening (or medical examination). The examination type information 21A is created based on various kinds of medical information such as clinical data, papers (monographs), books, electronic health records, database, and the like. The examination type information 21A can be reconstructed or updated when new medical information is acquired or the like. The reconstruction or update of the examination type information 21A is executed by the medical support system 1 or other computers.

As shown in FIG. 3, the examination type information 21A includes glaucoma, age-related macular degeneration (AMD), and cataract as examples of target diseases of screening. The examination type information 21A may further include other target diseases such as corneal endothelial disorder, diabetic retinopathy, occlusion of retinal vein, central serous chorioretinopathy, or pigmentary degeneration of retina. The present example describes information corresponding to glaucoma, age-related macular degeneration, and cataract. Information corresponding to other target diseases is given in the same manner.

The examination type information 21A includes the “DISEASE” section, the “HISTORY TAKING (ATTENTION WORDS)” section, the “FIRST EXAMINATION” section, the “SECOND EXAMINATION” section, AND the “THIRD EXAMINATION” section. In the disease section, the names of the target diseases of screening are recorded.

In the history taking section, character strings included in answers of patients obtained by history taking (e.g., chief complaint, present history, past history, family history, life history, occupational history, travel history), character strings possibly included in the answers, character strings resembling the character strings described above, character strings related to the character strings described above, and the like are recorded. For example, in the history taking section corresponding to glaucoma, character strings such as “FAMILY HISTORY” “FOGGY” “CLOUDY” “RAINBOW” “RING OF LIGHT”, “EYE PAIN”, “HEADACHE”, “HYPEREMIA”, “PART OF VISUAL FIELD IS DIFFICULT TO SEE”, and the like are recorded. In the history taking section corresponding to age-related macular degeneration, character strings such as “OLD AGE (OVER 50)”, “FAMILY HISTORY”, “SMOKING”, “FAR-SIGHTED”, “HIGH BLOOD PRESSURE”, “HIGH CHOLESTEROL”, “FEMALE”, “DISTORTION”, “COLOR DISCRIMINATION IS DIFFICULT”, “LOW VISION”, and the like are recorded. In the history taking section corresponding to cataract, character strings such as “BLUR”, “GLARING/DAZZLING”, “DOUBLE VISION”, “DIFFICULT TO SEE IN LIGHTED PLACE”, “SPECTACLE LENS IS NOT SUITABLE”, and the like are recorded.

In the first examination section, the first examination type group that are options of examination types for the first examination is recorded. For example, in the first examination section corresponding to glaucoma, visual acuity test, refractometry, tonometry, and fundus imaging (color) and the like are recorded. In addition, as the analysis of images (i.e., color fundus images) acquired by the fundus imaging (or fundus photography), optic nerve head (ONH) shape analysis, detection of optic nerve head bleeding, detection of nasal displacement of optic nerve head blood vessel, detection of peripapillary chorioretinal atrophy, detection of defect of retinal nerve fiber layer (RNFL), and the like are recorded. In the first examination section corresponding to age-related macular degeneration, fundus imaging (color, FAF) and the like are recorded. In addition, as the analysis of color fundus images, detection of geographic atrophy, detection of drusens (e.g., soft drusens, reticular pseudodrusens), detection of pigmentation, detection of serous retinal pigment epithelium (RPE) detachment, and the like are recorded. Further, as the analysis of autofluorescence fundus images (i.e., FAF images), detection of hypofluorescence at atrophy border, hyperfluorescence near atrophy, and the like are recorded. In the first examination section corresponding to cataract, fundus imaging (color) and the like are recorded. In addition, as the analysis of color fundus images, image quality analysis (brightness, color, contrast, sharpness) and the like are recorded.

In the second examination section, the second examination type group that are options of examination types for the second examination is recorded. For example, in the second examination section corresponding to glaucoma, fundus OCT and the like are recorded. In addition, as the analysis of images acquired by the fundus OCT, three dimensional shape analysis of optic nerve head (ONH), retinal nerve fiber layer (RNFL) thickness analysis, and the like are recorded. In the second examination section corresponding to age-related macular degeneration, fundus OCT and the like are recorded. In addition, as the analysis of images acquired by the fundus OCT, retinal pigment epithelium (RPE) thickness analysis, segment analysis (e.g., analysis of photoreceptor inner segment and outer segment junction (IS/OS) line, cone outer segment tip (COST) line, outer granular layer, external limiting membrane, choroid), and the like are recorded. In the second examination section corresponding to cataract, diagnosis by a specialist (i.e., consulting a specialist) is recorded. The specialist's diagnosis includes examination using a slit lamp microscope and visual acuity test.

In the third examination section, the third examination type group that are options of examination types for the third examination is recorded. For example, in the third examination section corresponding to glaucoma, diagnosis by a specialist (i.e., consulting a specialist) is recorded. The specialist's diagnosis includes tonometry, fundus photography, OCT, visual field test, and the like. In the third examination section corresponding to age-related macular degeneration, diagnosis by a specialist is recorded. The specialist's diagnosis includes color fundus imaging, autofluorescence photography, fluorescein angiography (FA), indocyanine green angiography (ICGA), OCT, and the like.

As described above, the examination type information 21A is table information in which various information is classified according to target diseases. The forms of the information for selecting examination types are not limited to the present example. For instance, when the artificial intelligence engine executes selection of examination types (e.g., at least one of the steps S3, S7, and S11 in FIG. 1), the examination type information 21 shown in FIG. 2 may include at least one of: information that can be referred to by technique disclosed in at least any one of the documents cited above; and information that can be referred to by any other known technique. Examples of artificial intelligence technology that can be applied to the present embodiment include neural network, deep learning, support vector machine, Bayesian network, association rule learning, reinforcement learning, feature learning (or representation learning), data mining, natural language processing, automated reasoning, and the like. The examination type information 21 may include dictionaries (e.g., medical dictionaries), corpora (e.g., medical corpora), knowledge bases (e.g., medical knowledge bases), or the like.

### <Data Reception Unit 40>

The data reception unit 40 receives the history data obtained by the history taking of the patient in the step S2 (Input history data) in FIG. 2. In addition, the data reception unit 40 receives the examination data acquired using the examination apparatuses in the step S6 (Input first examination data) and the step S10 (Input second examination data).

When answers obtained by history taking are entered into a computer (e.g., the history taking terminal 200) that can communicate with the medical support system 1, the data reception unit 40, for example, includes a communication device that receives the history data from the history taking terminal 200. The communication device may be included in the communication unit 50.

The history taking terminal 200 is, for example, a tablet computer, a desktop computer, a notebook computer, a dedicated computer, or the like.

When answers obtained by history taking are entered in a form (e.g., paper sheet), the data reception unit 40, for example, includes an image scanner that reads information recorded on the form, and an optical character recognition (OCR) processor that transforms the information (i.e., image) obtained by the image scanner into character codes. The optical character recognition processor includes a processor that operates according to optical character recognition software.

When the medical support system 1 includes the user interface 100 and when answers obtained by history taking are entered using the user interface 100, the data reception unit 40 is included in the user interface 100.

When the examinations are carried out using the examination apparatus 300 that can communicated with the medical support system 1, the data reception unit 40 includes, for example, a communication device that receives examination data from the examination apparatus 300. The communication device is included in the communication unit 50. The same applies to the case where examination data is input by way of another computer.

The examination apparatus 300 includes a plurality of ophthalmic examination apparatuses, for example. As a specific example, the examination apparatus 300 includes one or more fundus cameras (non-mydriatic fundus cameras), one or more OCT apparatuses, one or more visual acuity test apparatuses, one or more objective refractometers, one or more tonometers, and the like.

When examination data recorded in a recording medium is entered into the medical support system 1, the data reception unit 40 includes, for example, a data reader that reads out the examination data from the recording medium.

When examination data (e.g., measured values) is entered in a form, the data reception unit 40, for example, includes an image scanner that reads the examination data recorded on the form, and an optical character recognition processor that transforms the examination data (i.e., image) obtained by the image scanner into character codes.

When the medical support system 1 includes the user interface 100 and when examination data (e.g., measured values) is entered using the user interface 100, the data reception unit 40 is included in the user interface 100.

### <Communication Unit 50>

The communication unit 50 executes processing of transmitting data to other computers (e.g., the external computer 400) and processing of receiving data sent from the external computer 400. The communication unit 50 includes a known communication device in accordance with the communication system between the medical support system 1 and the external computer 400.

### <Data Processor 30>

The data processor 30 executes various kinds of processing. In the present example, the data processor 30 executes the step S3 (Select first examination type), the step S7 (Select second examination type), and the step S11 (Select third examination type) in FIG. 1. The data processor 30 includes the first selection unit 31 that executes the selection of first examination types, the second selection unit 32 that executes the selection of second examination types, and the third selection unit 33 that executes the selection of third examination types.

### <First Selection Unit 31>

The first selection unit 31 executes the step S3 (Select first examination type) in FIG. 1. More specifically, the first selection unit 31 selects one or more first examination types for the first examination from the examination type information 21 (e.g., from the first examination section of the examination type information 21A) based on the history data received by the data reception unit 40.

Examples of the processing executed by the first selection unit 31 will be described. The first selection unit 31 extracts character strings included in the history taking section of the examination type information 21A from the history data received by the data reception unit 40. The first selection unit 31 may be configured to extracts not only the same character strings as those included in the history taking section, but also character strings having the same meanings as those included in the history taking section, character strings related to those included in the history taking section, or the like.

Based on combinations of the character strings extracted from the history data, the first selection unit 31 selects one or more diseases that the patient may contract (referred to as possible diseases) from among the group of diseases (i.e., the disease names included in the disease section) included in the examination type information 21A. This processing can be executed by, for example, selecting a disease corresponding to a predetermined number or more (e.g., one or more) character strings among the extracted character strings.

In another example, for each of the disease names included in the disease section, the first selection unit 31 counts the number of character strings among the extracted character strings that are included in the history taking section corresponding to the disease name. In addition, the first selection unit 31 selects possible diseases based on the number of counts. For example, the first selection unit 31 may be configured to select a predetermined number of possible diseases (e.g., three possible diseases) in decreasing order of the number of counts. In this case, for each of the possible diseases, the first selection unit 31 can calculate the probability that the patient contracts the possible disease based on the number of counts (referred to as morbid probability). Here, the relationship between numbers of counts and morbid probabilities can be determined based on clinical data, medical knowledge bases, and the like. The first selection unit 31 may also be configured to calculate morbid probability by assigning a weight to each of the character strings included in the history taking section, and by calculating the weighted sum of the plurality of character strings counted.

In yet another example, the first selection unit 31 includes an artificial intelligence engine, medical knowledge bases, or the like to determine disease names.

Next, for each of the selected diseases, the first selection unit 31 specifies the first examination section corresponding to the selected disease, and determines one or more first examination types included in the specified first examination section.

It is also possible to execute the selection of first examination types from history data without referring to disease names. For example, an artificial intelligence engine, medical knowledge bases, or the like can be utilized for the automated reasoning to find first examination types based on history data (that is, based on character strings etc. included in history data).

### <Second Selection Unit 32>

The second selection unit 32 executes the step S7 (Select second examination type) in FIG. 1. More specifically, the second selection unit 32 selects one or more second examination types for the second examination from the examination type information 21 (e.g., from the second examination section of the examination type information 21A) based on the history data received by the data reception unit 40 and the first examination data acquired by the first examination.

In this manner, the second selection unit 32 executes the processing in response to the reception of the result obtained by the first selection unit 31. Hence, it is assumed that the meaning of the procedure for selecting second examination types based on history data and first examination data also includes the procedure for selecting second examination types based on first examination data without history data.

Examples of the processing executed by the second selection unit 32 will be described. Based on the history data (e.g., the history data itself, the character strings extracted from the history data by the first selection unit 31, the diseases selected by the first selection unit 31, or the like) and the first examination data, the second selection unit 32 selects one or more second examination types included in the second examination section in the examination type information 21A.

The first examination data can be processed by means of the second selection unit 32, a component of the data processor 30 other than the second selection unit 32, or the external computer 400. For example, an artificial intelligence engine can be employed to process the first examination data. In a typical example, the first examination data includes image data that represents the structure or the function of a predetermined site of the patient. Color fundus images and OCT images are examples of the image data representing the structure. Fluorescence fundus angiograms and OCT blood flow images are examples of the image data representing the function. When fundus imaging is carried out in the first examination, a fundus image (image data) of the patient is obtained as the first examination data. The artificial intelligence engine can process the fundus image included in the first examination data to determine possible diseases, morbid probabilities, second examination types, or the like. Alternatively, the artificial intelligence engine can process the fundus image included in the first examination data to acquire information for determining possible diseases, morbid probabilities, second examination types, or the like.

Based on the history data (e.g., the history data itself, the character strings extracted from the history data by the first selection unit 31, the diseases selected by the first selection unit 31, or the like) and the result of the process of the image data acquired by the artificial intelligence engine, the second selection unit 32 executes the selection of second examination types. As in the case of the first selection unit 31, the second selection unit 32 can also determine possible diseases, morbid probabilities, or the like.

The processing of selecting second examination types based on history data and first examination data is not limited to the above processing. For example, it is possible to introduce an artificial intelligence engine, medical knowledge bases, or the like to execute automated reasoning for determining second examination types based on history data and first examination data.

### <Third Selection Unit 33>

The third selection unit 33 executes the step S11 (Select third examination type) in FIG. 1. More specifically, the third selection unit 33 selects one or more third examination types for the third examination from the examination type information 21 (e.g., from the third examination section of the examination type information 21A) based on the history data received by the data reception unit 40, the first examination data acquired by the first examination, and the second examination data acquired by the second examination.

In this manner, the third selection unit 33 executes the processing in response to the reception of the result obtained by the second selection unit 32. Hence, it is assumed that the meaning of the procedure for selecting third examination types based on history data, first examination data, and the second examination data also includes the procedure for selecting third examination types based on first examination data and the second examination data without history data and the procedure for selecting third examination types based on the second examination data without both history data and first examination data.

Examples of the processing executed by the third selection unit 33 will be described. Based on the history data (e.g., the history data itself, the character strings extracted from the history data by the first selection unit 31, the diseases selected by the first selection unit 31, or the like), the first examination data, and the second examination data, the third selection unit 33 selects one or more third examination types included in the third examination section in the examination type information 21A.

The second examination data (and the first examination data) can be processed by means of the third selection unit 33, a component of the data processor 30 other than the third selection unit 33, or the external computer 400. For example, an artificial intelligence engine can be employed to process the second examination data. For example, when fundus OCT is carried out in the second examination, an OCT image (image data) is acquired. An artificial intelligence engine can process the OCT image included in the second examination data to determine possible diseases, morbid probabilities, third examination types, or the like. Alternatively, the artificial intelligence engine can process the OCT image included in the second examination data to acquire information for determining possible diseases, morbid probabilities, third examination types, or the like.

Based on the history data (e.g., the history data itself, the character strings extracted from the history data by the first selection unit 31, the diseases selected by the first selection unit 31, or the like), the result of the process of the first examination data (e.g., fundus image) acquired by the artificial intelligence engine, and the result of the process of the second examination data (e.g., OCT image) acquired by the artificial intelligence engine, the third selection unit 33 executes the selection of third examination types. As in the case of the first selection unit 31, the third selection unit 33 can also determine possible diseases, morbid probabilities, or the like.

The processing of selecting third examination types based on history data, first examination data, and second examination data is not limited to the above processing. For example, it is possible to introduce an artificial intelligence engine, medical knowledge bases, or the like to execute automated reasoning for determining third examination types based on history data, first examination data, and second examination data.

### <User Interface 100>

The user interface 100 includes the display unit 101 and the operation unit 102. The display unit 101 includes a display device such as a flat panel display. The operation unit 102 includes operation devices such as a mouse, a keyboard, a trackpad, a button, a key, a joystick, or an operation panel.

The display unit 101 and the operation unit 102 may be a single device or may be separated devices. For example, a device having both the display function and the operation function, like a touch panel, can be employed. In such a case, the operation unit 102 includes the touch panel and a computer program. The content of an operation performed using the operation unit 102 is input into the controller 10 as an electrical signal. A graphical user interface (GUI) displayed on the display unit 101 and the operation unit 102 can be used for operation and input of information.

### <Usage Modes>

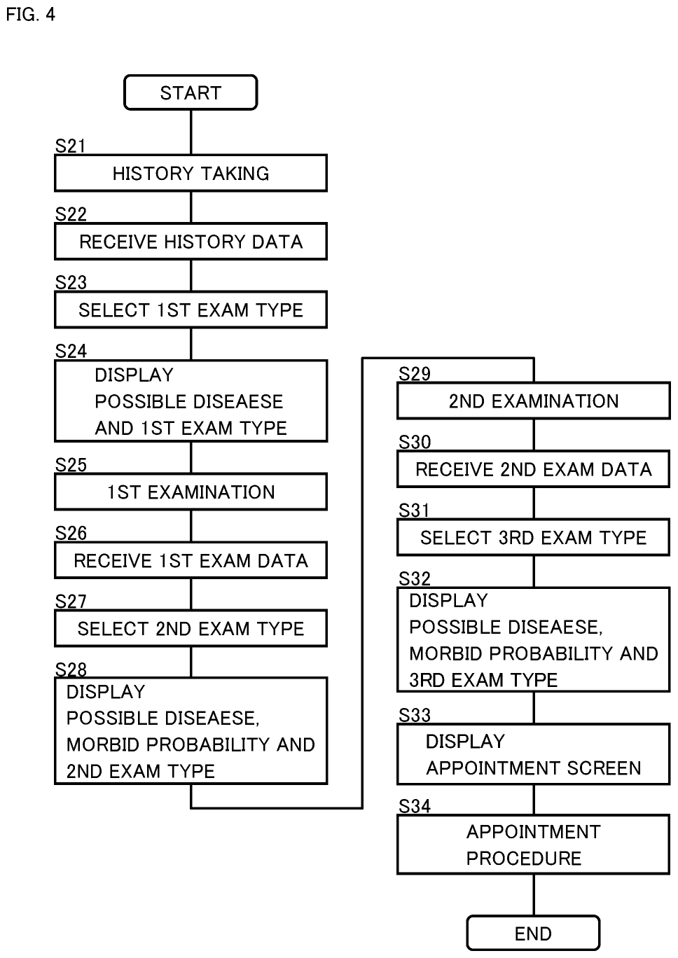

Usage modes that can be implemented by the medical support system 1 will be described. FIG. 4 shows an example of the usage mode.

### (S21: History Taking)

To begin with, at step 405 (also referred to herein as “S21”), history taking of the patient is carried out. The answers obtained from the patient are entered into the history taking terminal 200.

### (S22: Receive History Data)

The data reception unit 40 receives the history data, at step 410 (also referred to herein as “S22”), entered into the history taking terminal 200 in the step S21.

### (S23: Select First Examination Type)

The first selection unit 31 executes any of the processing described above to select one or more first examination types, at step 415 (also referred to herein as “S23”), for the first examination from the first examination type group included in the examination type information 21 (e.g., from the first examination section of the examination type information 21A) based on the history data received by the data reception unit 40. In addition, the first selection unit 31 can determine possible diseases (or candidate diseases), morbid probabilities, or the like.

### (S24: Display Possible Disease, First Examination Type, Etc.)

The output controller 11 controls the display device 101, at step 420 (also referred to herein as “S24”), to display information based on the possible diseases (and their morbid probabilities) and the first examination types determined by the first selection unit 31. Note that the history data is the only material to be processed at this stage; therefore, calculation and display of morbid probability is not necessary.

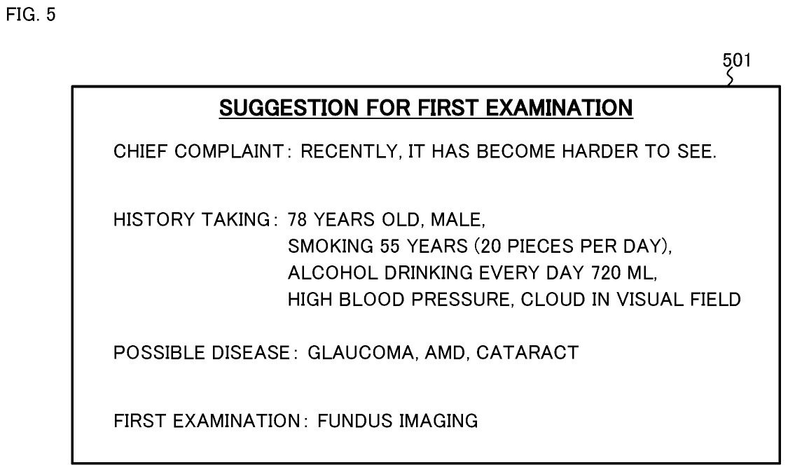

FIG. 5 shows an example of the information displayed in the step S24. The display screen 501 illustrated in FIG. 5 presents the chief complaint, at least part of the history data other than the chief complaint, the possible diseases, and the first examination types. The chief complaint and other information from the history data are extracted from the history data by the first selection unit 31. The possible diseases and the first examination types are determined (e.g., specified, inferred, deduced, reasoned, or the like) from the history data by the first selection unit 31.

The chief complaint section of the display screen 501 presents information “RECENTLY, IT HAS BECOME HARDER TO SEE.” that has been extracted from the history data. The history taking section presents information “78 YEARS OLD, MALE, SMOKING 55 YEARS (20 PIECES PER DAY), ALCOHOL DRINKING EVERY DAY 720 ML, HIGH BLOOD PRESSURE, CLOUD IN VISUAL FIELD” that has been extracted from the history data. The possible disease section presents the names of the possible diseases “GLAUCOMA, AMD, CATARACT” that has been reasoned based on the history data by the first selection unit 31. The first examination section presents the examination type (examination item) “FUNDUS IMAGING” that has been reasoned based on the history data by the first selection unit 31.

### (S25: First Examination)

Based on the first examination types displayed on the display unit 101, the first examination on the patient is carried out at step 425 (also referred to herein as “S25”). The first examination is carried out using any of the examination apparatus 300. In the case shown in FIG. 5, fundus imaging of one eye or both eyes of the patient is carried out using the (non-mydriatic) fundus camera. The fundus imaging is, for example, color fundus photography only, or both color fundus photography and FAF. In addition, analysis of the first examination data, such as the analysis of the fundus image acquired by the fundus imaging, can be executed.

### (S26: Receive First Examination Data)

The data reception unit 40 receives the first examination data, at step 430 (also referred to herein as “S26”), obtained by the examination apparatus 300 in the first examination.

### (S27: Select Second Examination Type)

The second selection unit 32 executes any of the processing described above to select one or more second examination types, at step 435 (also referred to herein as “S27”), for the second examination from the second examination type group included in the examination type information 21 (e.g., from the second examination section of the examination type information 21A) based on the history data and the first examination data received by the data reception unit 40. In addition, the second selection unit 32 can determine possible diseases (or candidate diseases), morbid probabilities, or the like.

### (S28: Display Possible Disease, Morbid Probability, Second Examination Type, Etc.)

The output controller 11 controls the display device 101 to display information, at step 440 (also referred to herein as “S28”), based on the possible diseases, their morbid probabilities, and the second examination types determined by the second selection unit 32.

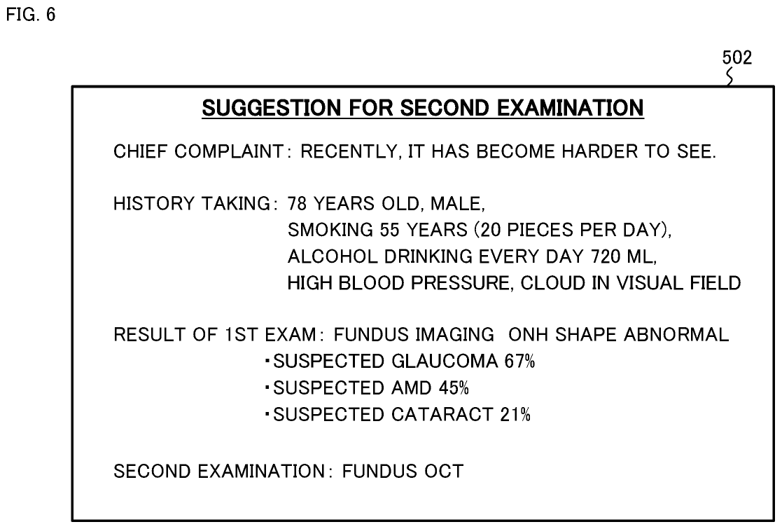

FIG. 6 shows an example of the information displayed in the step S28. The display screen 502 illustrated in FIG. 6 presents, as in the case of the display screen 501 illustrated in FIG. 5, the chief complaint and other history data. In addition, the result of first examination section in the display screen 502 presents the fact that fundus imaging has been carried out for the first examination, the analysis result that the shape of the optic nerve head (ONH) may be abnormal, the morbid probability for glaucoma (i.e., the probability of the suspicion of glaucoma) is 67 percent, the morbid probability for age-related macular degeneration is 45 percent, and the morbid probability for cataract is 21 percent. The morbid probabilities have been reasoned by the second selection unit 32 based on the history data and the first examination data. Further, the second examination section presents the examination type “FUNDUS OCT” that has been reasoned by the second selection unit 32 based on the history data and the first examination data.

### (S29: Second Examination)

Based on the second examination types displayed on the display unit 101, the second examination on the patient is carried out at step 445 (also referred to herein as “S29”). The second examination is carried out using any of the examination apparatus 300. In the case shown in FIG. 6, fundus OCT of one eye or both eyes of the patient is carried out using the OCT apparatus. In addition, analysis of the second examination data, such as the analysis of the OCT image acquired by the fundus OCT, can be executed.

### (S30: Receive Second Examination Data)

The data reception unit 40 receives the second examination data, at step 450 (also referred to herein as “S30”), obtained by the examination apparatus 300 in the second examination.

### (S31: Select Third Examination Type)

The third selection unit 33 executes any of the processing described above to select one or more third examination types, at step 455 (also referred to herein as “S31”), for the third examination from the third examination type group included in the examination type information 21 (e.g., from the third examination section of the examination type information 21A) based on the history data, the first examination data, and the second examination data received by the data reception unit 40. In addition, the third selection unit 33 can determine possible diseases, their morbid probabilities, or the like.

### (S32: Display Possible Disease, Morbid Probability, Third Examination Type, Etc.)

The output controller 11 controls the display device 101 to display information, at step 460 (also referred to herein as “S32”), based on the possible diseases, their morbid probabilities, and the third examination types determined by the third selection unit 33.

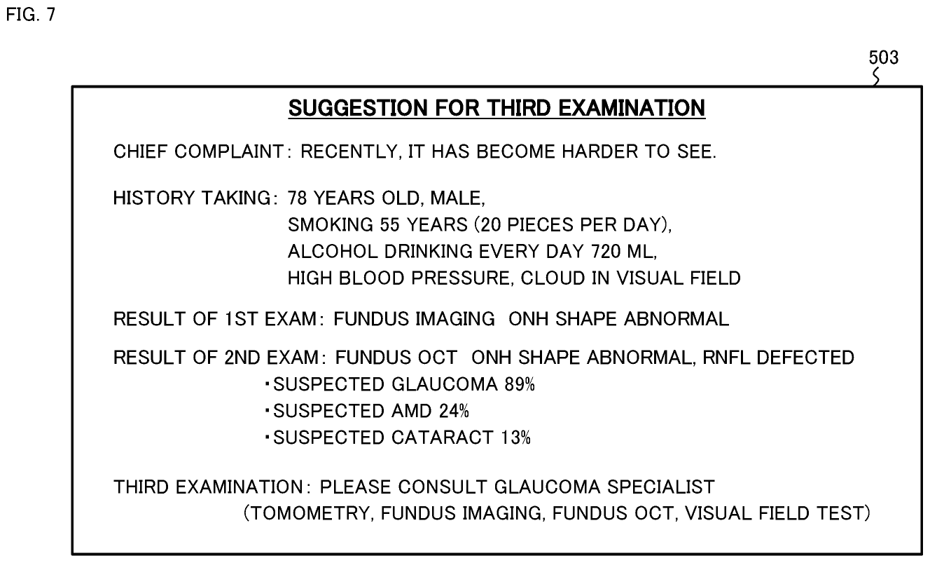

FIG. 7 shows an example of the information displayed in the step S32. The display screen 503 illustrated in FIG. 7 presents, as in the case of the display screen 502 illustrated in FIG. 6, the chief complaint, other history data, and the result of the first examination. In addition, the result of second examination section in the display screen 503 presents the fact that fundus OCT has been carried out for the second examination, the analysis result that the shape of the optic nerve head (ONH) may be abnormal, the analysis result that the retinal nerve fiber layer (RNFL) may be defected, the morbid probability for glaucoma is 89 percent, the morbid probability for age-related macular degeneration is 24 percent, and the morbid probability for cataract is 13 percent. The morbid probabilities have been reasoned by the third selection unit 33 based on the history data, the first examination data, and the second examination data. Further, the third examination section presents information “PLEASE CONSULT GLAUCOMA SPECIALIST (TONOMETRY, FUNDUS IMAGING, FUNDUS OCT, VISUAL FIELD TEST)” that has been determined by the third selection unit 33 based on the history data, the first examination data and the second examination data.

As can be seen from the comparison between the result of the first examination shown in FIG. 6 and the result of the second examination shown in FIG. 7, the morbid probability for glaucoma in the second examination result is higher than that in the first examination result, and the morbid probabilities for age-related macular degeneration and for cataract in the second examination result both are lower than those in the first examination result. This means that possible diseases (candidate diseases) has been narrowed by taking the second examination data into account in addition to the history data and the first examination data. That is, taking the second examination data into account in addition to the history data and the first examination data improves the accuracy (precision) of the reasoning of possible diseases (candidate diseases).

### (S33: Display Appointment Screen)

When the consultation with a specialist is suggested as in the case of FIG. 7, the controller 10 starts up application software for appointment to consult a specialized medical institution or an advanced medical institution. The output controller 11 controls the display unit 101 to display the appointment screen at step 465 (also referred to herein as “S33”).

As described above, the appointment screen presents, for example, patient information, the list of appointable medical institutions, objects for selecting whether the patient hopes to make an appointment or not, objects for designating preferred appointment dates, appointable dates of the respective medical institutions, and the like.

### (S34: Appointment Procedure)

The user (e.g., patient, examiner) enters necessary information, at step 470 (also referred to herein as “S34”), into the appointment screen using the operation unit 102. The entered information is sent to the in-hospital server of the medical institution designated. When the appointment processing is completed, information indicating the completion of appointment is transmitted from the in-hospital server to the medical support system 1, the patient's address, or the like.

### <Actions and Effects>

The actions and the effects according to the embodiment described above will be described.

A medical support method of an embodiment is implemented by using a computer. the computer performs the following steps. Firstly, the computer receives history data obtained by carrying out history taking of a patient. Then, based on the history data, the computer selects one or more first examination types for a first examination from a first examination type group stored in a storage device in advance. Then, the computer controls an output device to output information indicating the one or more first examination types selected. After the patient undergoes the first examination that includes at least one of the one or more first examination types, the computer receives first examination data obtained from the first examination of the patient. Then, based on the history data and the first examination data, the computer selects one or more second examination types for a second examination from a second examination type group stored in the storage device in advance. Then, the computer controls the output device to output information indicating the one or more second examination types selected.

Such a medical support method can be implemented, for example, with the exemplary medical support system described above. The medical support system 1 includes the storage unit 20, the data reception unit 40, the first selection unit 31, the display unit 101, the output controller 11, and the second selection unit 32. Note that the display unit 101 may be arranged outside the medical support system 1.

The storage unit 20 functions as the storage device, and is configured to store the first examination type group and the second examination type group in advance. The data reception unit 40 functions as the history data reception unit, and is configured to the receive history data obtained from the history taking of the patient. The first selection unit 31 is configured to select one or more first examination types for the first examination from the first examination type group based on the history data. The display unit 101 functions as the output unit. Note that the output mode of the output unit is not limited to display, and may be audio output, print, data transmission, data writing into recording media, or the like. The output controller 11 is configured to control the display unit 101 to display the information indicating the one or more first examination types. After the patient undergoes the first examination that includes at least one of the one or more first examination types, the data reception unit 40 receives the first examination data obtained from the first examination. In the present example, the data reception unit 40 functions as both the history data reception unit and the examination data reception unit. The second selection unit 32 is configured to select one or more second examination types for the second examination from the second examination type group based on the history data and the first examination data. The output controller 11 is configured to control the display unit 101 to display the information indicating the one or more second examination types selected.

According to the exemplary method and system for medical support, examination types for the first examination can be determined based on the history data, and examination types for the second examination can be determined by taking into account the first examination data obtained from the first examination in addition to the history data. Hence, it is possible to selectively and stepwisely carry out examinations as may be necessary for medical screening or the like. As a result, time and burdens required for examinations can be decreased, burdens on the patient can be decreased, burdens on medical workers can be decreased, and efficiency of diagnosis and treatment can be improved. The method and the system of the present embodiment are effective not only in the case where examinations are carried out under the management of a medical specialist or the like, but also in the case where examinations are carried out in a location in which no medical specialist exists such as home healthcare or medical checkup vehicles. In this manner, according to the method and the system of the present embodiment, it is possible to decrease burdens on patients and examiners and to improve the efficiency of diagnosis and treatment while maintaining accuracy of diagnosis.

Further features, actions, and effects of the above exemplary embodiment will be described.

The computer (the second selection unit 32) may be configured to select one or more possible diseases (or candidate diseases) of the patient from a disease group stored in the storage device (the storage unit 20) in advance based on the history data and the first examination data, and select the one or more second examination types based on the one or more possible diseases selected.

In addition, the computer (the output controller 11) may be configured to control the output device (the display unit 101) to output the information indicating the possible diseases and the information indicating the second examination types.

With such configurations, it is possible to give information on the possible diseases together with the second examination types to the patient, the examiner, or the like.

The computer (the second selection unit 32) may be configured to determine the morbid probability for each of the possible diseases based on the history data and the first examination data, and select the second examination types based on the possible diseases and their morbid probabilities.

In addition, the computer (the output controller 11) may be configured to control the output device (the display unit 101) to output the information indicating the possible diseases, the information indicating the morbid probabilities, and the information indicating the second examination types.

With such configurations, it is possible to give information on the possible diseases and their morbid probabilities together with the second examination types to the patient, the examiner, or the like.

the patient undergoes the second examination that includes at least one of the one or more second examination types, the computer (the data reception unit 40) can receive the second examination data obtained from the second examination of the patient. Then, the computer (the third selection unit 33) can select one or more third examination types for the third examination from the third examination type group stored in the storage device (the storage unit 20) in advance based on the history data, the first examination data, and the second examination data. Then, the computer (the output controller 11) can control the output device (the display unit 101) to output information indicating the one or more third examination types selected.

With such a configuration, examination types for the first examination can be determined based on the history data, examination types for the second examination can be determined by taking into account the first examination data obtained from the first examination in addition to the history data, and examination types for the third examination can be determined by taking into account the second examination data obtained from the second examination in addition to the history data and the first examination data. Hence, it is possible to carry out examinations as may be necessary for medical screening or the like in more stepwise fashion. As a result, the effects of the method and the system of the present embodiment can further be improved.

Moreover, the computer (the third selection unit 33) may be configured to select one or more possible diseases of the patient from a disease group stored in the storage device (the storage unit 20) in advance based on the history data, the first examination data, and the second examination data, and select the third examination types based on the selected possible diseases.

In addition, the computer (the output controller 11) controls the output device (the display unit 101) to output information indicating the selected possible diseases and the information indicating the third examination types.

With such configurations, it is possible to give information on the possible diseases together with the third examination types to the patient, the examiner, or the like.

Moreover, the computer may be configured to determine the morbid probability for each of the possible diseases based on the history data, the first examination data, and the second examination data, and select the third examination types based on the possible diseases and their morbid probabilities.

In addition, the computer (the output controller 11) controls the output device (the display unit 101) to output the information indicating the possible diseases, the information indicating the morbid probabilities, and the information indicating the third examination types.

With such configurations, it is possible to give information on the possible diseases and their morbid probabilities together with the third examination types to the patient, the examiner, or the like.

The computer may include an artificial intelligence engine. For example, at least one of the first selection unit 31, the second selection unit 32, and the third selection unit 33 may be configured to function as an artificial intelligence engine. Any two or more of the first selection unit 31, the second selection unit 32, and the third selection unit 33 may be configured with the same hardware (e.g., processor). In another example, an artificial intelligence engine may be included in other components of the data processor 30. In yet another example, an artificial intelligence engine may be included in an apparatus arranged outside the medical support system 1 (i.e., in the external computer 400). When an artificial intelligence engine is employed, data and/or database for the processing of the artificial intelligence engine is stored in the storage device (storage unit 20) or the external computer 400, or in a storage device accessible by the medical support system 1 or the external computer 400.

When the history data includes a natural language (i.e., information represented with a natural language), the artificial intelligence engine (e.g., the first selection unit 31, the second selection unit 32, and the third selection unit 33, the data processor 30, the external computer 400) can apply natural language processing to the history data. The first selection unit 31 can executes the selection of first examination types (and the selection of possible diseases and/or the calculation of morbid probabilities) based on the result of the natural language processing. Similarly, the second selection unit 32 can executes the selection of second examination types (and the selection of possible diseases and/or the calculation of morbid probabilities) based on the result of the natural language processing. In addition, the third selection unit 33 can executes the selection of third examination types (and the selection of possible diseases and/or the calculation of morbid probabilities) based on the result of the natural language processing.

When the first examination data includes image data that represents the structure or the function of a predetermined site of the patient (e.g., fundus image), the artificial intelligence engine (e.g., the first selection unit 31, the second selection unit 32, and the third selection unit 33, the data processor 30, the external computer 400) can execute processing based on the image data. Examples of the processing includes image quality improvement, segmentation, feature extraction, lesion detection, data mining, and the like. The second selection unit 32 can performs the selection of second examination types based on the result obtained by the artificial intelligence engine.

In the same manner, when the second examination data includes image data that represents the structure or the function of a predetermined site of the patient (e.g., OCT image), the artificial intelligence engine (e.g., the first selection unit 31, the second selection unit 32, and the third selection unit 33, the data processor 30, the external computer 400) can execute processing based on the image data. Examples of the processing includes image quality improvement, segmentation, feature extraction, lesion detection, data mining, and the like. The third selection unit 33 can performs the selection of third examination types based on the result obtained by the artificial intelligence engine.

With such artificial intelligence engines, it becomes possible to perform the selection (or reasoning) of examination types, the selection (or reasoning) of possible diseases, the calculation of morbid probabilities, and the like with better precision and better accuracy.

The computer (the output controller 11) may be configured to control the display device (the display unit 101) to display a screen for medical care appointment (appointment screen) based on the history data and the first examination data or based on the history data, the first examination data and the second examination data. The computer (the user interface 100, the operation unit 102) can receive the information input to the appointment screen. The computer may be configured to perform processing for medical care appointment of the patient based on the received information.

With such a configuration, it is possible to easily and smoothly carry out appointment procedure when the consultation with a medical specialist is required or the like.

Processes included in medical support methods according to embodiments are not limited to those in the examples described above. Similarly, components (configuration, actions, operations, etc.) included in medical support systems according to embodiments are not limited to those in the examples described above.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A system for supporting an ophthalmic clinical examination using a computer, wherein the ophthalmic clinical examination includes ophthalmic measurement and/or ophthalmic imaging, the system comprising:
circuitry configured as a memory that retains a first examination type group including a plurality of first examination types and a second examination type group including a plurality of second examination types, each of the plurality of first examination types is a type of the ophthalmic measurement or a type of the ophthalmic imaging, and each of the plurality of second examination types is a type of the ophthalmic measurement or a type of the ophthalmic imaging;
processor circuitry configured as a first selecting processor that selects one or more first examination types from the first examination type group based on medical interview data of a patient;
the processor circuitry further configured as a first output controlling processor that controls a first output device to output information on the one or more first examination types selected by the first selecting processor;
the processor circuitry further configured as a second selecting processor that selects one or more second examination types from the second examination type group, based on the medical interview data and on first examination data acquired from the patient by a first examination conducted on the basis of the one or more first examination types selected by the first selecting processor; and
the processor circuitry further configured as a second output controlling processor that controls a second output device to output information on the one or more second examination types selected by the second selecting processor.

2. The system of claim 1, wherein
the memory further retains a disease group including a plurality of ophthalmic disease names, and
the second selecting processor selects one or more ophthalmic disease names from the disease group based on the medical interview data and on the first examination data, and selects one or more second examination types from the second examination type group based on the one or more ophthalmic disease names selected.

3. The system of claim 2, wherein the second output controlling processor controls the second output device to output information on the one or more ophthalmic disease names selected by the second selecting processor and the information on the one or more second examination types.

4. The system of claim 2, wherein the second selecting processor estimates one or more morbid probabilities respectively corresponding to the one or more ophthalmic disease names selected from the disease group based on the medical interview data and on the first examination data, and selects one or more second examination types from the second examination type group based on the one or more ophthalmic disease names and on the one or more morbid probabilities.

5. The system of claim 4, wherein the second output controlling processor controls the second output device to output information on the one or more ophthalmic disease names selected by the second selecting processor, information on the one or more morbid probabilities estimated by the second selecting processor, and the information on the one or more second examination types.

6. The system of claim 1, wherein the memory further retains a third examination type group including a plurality of third examination types, the system further comprising:
the processor circuitry further configured as a third selecting processor that selects one or more third examination types from the third examination type group, based on the medical interview data, on the first examination data, and on second examination data acquired from the patient by a second examination conducted on the basis of the one or more second examination types selected by the second selecting processor; and
the processor circuitry further configured as a third output controlling processor that controls a third output device to output information on the one or more third examination types selected by the third selecting processor.

7. The system of claim 6, wherein the memory further retains a disease group including a plurality of ophthalmic disease names, and
the third selecting processor selects one or more ophthalmic disease names from the disease group based on the medical interview data, on the first examination data, and on the second examination data, and selects one or more third examination types from the third examination type group based on the one or more ophthalmic disease names selected.

8. The system of claim 7, wherein the third output controlling processor controls the third output device to output information on the one or more ophthalmic disease names selected by the third selecting processor and the information on the one or more third examination types.

9. The system of claim 7, wherein the third selecting processor selects one or more morbid probabilities respectively corresponding to the one or more ophthalmic disease names selected from the disease group based on the medical interview data, on the first examination data, and on the second examination data, and selects one or more third examination types from the third examination type group based on the one or more ophthalmic disease names and on the one or more morbid probabilities.

10. The system of claim 9, wherein the third output controlling processor controls the third output device to output information on the one or more ophthalmic disease names selected by the third selecting processor, information on the one or more morbid probabilities estimated by the third selecting processor, and the information on the one or more third examination types.

11. The system of claim 1, wherein the processor circuitry includes hardware and software that function as an artificial intelligence engine.

12. The system of claim 11, wherein
the medical interview data includes natural language information input using a natural language, and
the first selecting processor performs a natural language process on the medical interview data using the artificial intelligence engine, and selects one or more first examination types from the first examination type group based on information acquired by the natural language process.

13. The system of claim 11, wherein
the medical interview data includes natural language information input using a natural language, and
the second selecting processor performs a natural language process on the medical interview data using the artificial intelligence engine, and selects one or more second examination types from the second examination type group based on information acquired by the natural language process and on the first examination data.

14. The system of claim 11, wherein
the first examination data includes image data representing a structure or a function of an eye of the patient, and
the second selecting processor performs a process on the image data using the artificial intelligence engine, and selects one or more second examination types from the second examination type group based on information acquired by the process performed on the image data and on the medical interview data.

15. The system of claim 1, further comprising
the processor circuitry further configured as a display controlling processor controls a display device to display a screen for medical care appointment of a medical institution based at least on the medical interview data and the first examination data;
an input device used for an operation of inputting information to the screen displayed on the display device; and
the processor circuitry further configured as an appointment processor that performs a process of medical care appointment for the patient based on the information input to the screen using the input device.

16. The system of claim 1, further comprising a first examination device that is connected to and communicate with the computer and that acquires the first examination data from the patient by the first examination.

17. The system of claim 6, further comprising a second examination device that is connected to and communicate with the computer and that acquires the second examination data from the patient by the second examination.
